(19) United States
(12) Patent Application Publication
(10) Pub. No.: US 2016/0228531 A1
(43) Pub. Date: Aug. 11, 2016

(54) TREATMENT OF HEPATITIS D VIRUS INFECTIONS BY REDIRECTION OF T CELLS

(71) Applicant: CHRONTECH PHARMA AB, Huddinge (SE)

(72) Inventors: Matti Sällberg, Stockholm (SE); Lars Frelin, Tullinge (SE); Margaret Sällberg Chen, Stockholm (SE); Gustaf Ahlen, Järfälla (SE)

(21) Appl. No.: 15/022,896

(22) PCT Filed: Sep. 17, 2014
(86) PCT No.: PCT/US2014/056158

§ 371 Date: Mar. 17, 2016

Related U.S. Application Data
(60) Provisional application No. 61/879,881, filed on Sep. 19, 2013.

(51) Int. Cl. A61K 39/12; C12N 5/0783; A61K 45/06; C07K 14/725; A61K 35/17

(52) U.S. Cl. CPC A61K 39/12; C07K 14/7051; A61K 35/17; A61K 45/06; C12N 5/0636; A61K 2039/5158

(57) ABSTRACT

Several approaches to treat, inhibit, or prevent HDV infections by redirecting T cells to HDV and HDV infected cells are described. In several alternatives, a gene encoding a molecule that specifically binds to human leucocyte antigen (HLA) loaded with a HDV derived peptide are introduced into the T cells of individuals. Upon receiving the modified T cells, the modified T cells, derived from the individual, will then recognise the individual's cells that are infected by HDV and/or the HDV virus, blocking HDV replication and/or killing the HDV infected cells thereby preventing and/or treating or inhibiting the HDV infection in the individual.

### FIELD OF THE INVENTION

The disclosure herein relates to the field of immunology and, more specifically, the redirection of T cells to Hepatitis D virus (HDV) in infected individuals or individuals at risk of becoming infected with HDV.

### BACKGROUND OF THE INVENTION

Hepatitis is a disease resulting in swelling and inflammation of the liver. This disorder is commonly caused by viruses, five types of which are currently known (Hepatitis A, B, C, D and E). Hepatitis D virus (HDV), also referred to as Hepatitis delta virus, is a small, spherical single-stranded circular RNA virus. HDV is a highly pathogenic virus that causes acute, fulminant, and rapidly progressing chronic hepatitis. The entire virus was cloned and sequenced in 1986, and given the genus of Deltavirus. HDV is structurally unrelated to the other hepatitis viruses. Since HDV is an incomplete virus, it can only replicate in the presence of Hepatitis B (HBV) virus, which provides structural components for HDV. In particular, HDV has an outer coat that contains large, medium and small hepatitis B surface antigens, and host lipids surrounding an inner nucleocapsid, which contains about 200 molecules of hepatitis D antigen (HDAg) for each genome. The circular genome of HDV is unique to animal viruses because of its high GC content.

Chronic infections caused by the hepatitis B virus (HBV) and hepatitis D virus (HDV) are major causes for severe liver disease and liver cancer globally. Two billion people have been in contact with HBV and of these infected individuals, approximately 400 million are chronically infected, and of the chronically infected individuals approximately 15 million are co-infected with HDV. HDV is currently emerging in Europe due to immigration from high endemic areas. Within the European Community (EC), approximately 7 million people are believed to have chronic HBV infection and out of these infected individuals, approximately 5% (i.e., 350,000) are thought to be co-infected with HDV.

Potent antivirals inhibit HBV replication without affecting the HDV replication. This is due to the fact that these antivirals affect neither the production of the HBV envelope (HBsAg) required for HDV assembly, nor the replication of the HDV genome mediated by the host cell RNA polymerase II. The latter significantly impairs the possibility to develop antiviral enzyme inhibitors for HDV. HBsAg-based HBV vaccines can prevent a non-infected subject from becoming infected by both HBV and HDV; however, the HBV vaccine cannot protect a subject already infected by HBV against HDV super-infection due to the inherent overproduction of HBsAg during the HBV infection.

No specific treatment for hepatitis D virus (HDV) exists. The only treatment available for HDV today is an expensive and cumbersome 48 month long therapy using pegylated interferon (PEG-IFN), which only treats 25% of HDV infections. Accordingly, new preventive and therapeutic strategies are desperately needed for the increasing problem of HBV-HDV co-infections.

### BRIEF SUMMARY OF THE INVENTION

In the present disclosure, several approaches to treat, inhibit, ameliorate, or prevent HDV infections by redirecting T cells to HDV infected cells are described, in several alternatives, a gene (e.g., RNA or DNA) encoding a molecule that specifically binds to human leucocyte antigen (HLA)-A2 loaded with a HDV derived peptide are introduced into the T cells of individuals (e.g., patients that have been selected or identified as being infected with HDV or patients that are selected or identified as being at risk of becoming infected with HDV). Upon receiving the modified T cells, the modified T cells, derived from the patient or the donor, will then recognise the patient's cells that are infected by the HDV virus, and blocking HDV replication and/or killing the HDV infected cells thereby preventing and/or treating, ameliorating, or inhibiting the HDV infection in the patient. Accordingly, aspects of the invention relate to nucleic acids and peptides that are useful for the preparation of medicaments for the prevention, treatment, inhibition, or amelioration of HDV infection, as well as, methods of use thereof to redirect a patient's T cells to HDV and thereby prevent, treat, inhibit, or ameliorate HDV infection and diseases associated therewith including, but not limited to, chronic HBV-HDV co-infection, liver disease (e.g., HDV-mediated liver cirrhosis), and liver cancer (e.g., hepatocellular cancer (HCC)). These medicaments, methods and treatment, inhibitions, ameliorations, and/or prevention protocols can further include conventional HBV and/or HDV medicaments and/or therapies including, but not limited to, a peptide or DNA-based HBV vaccine (e.g., a HBsAg-based HBV vaccine) and/or PEG-IFN. Some of the alternatives described herein are provided in the following section.

Alternatives

1. An isolated nucleic acid comprising a nucleotide sequence that encodes an alpha and/or a beta peptide, which forms a part of or a complete T cell receptor, which binds a hepatitis D virus (HDV) antigen, wherein said HDV antigen is present on HDV or an HDV infected cell, and wherein T cells that express said T cell receptor inhibit HDV replication with or without killing the cell.

2. An isolated nucleic acid comprising a nucleotide sequence that encodes an alpha and/or a beta peptide, which forms a part of or a complete T cell receptor, which binds a hepatitis D virus (HDV) antigen, wherein said HDV antigen is present on HDV or an HDV infected cell, and wherein T cells that express said I cell receptor inhibit HDV replication with or without killing the cell through the interaction with the HLA-A2 molecule loaded with a peptide from the HDV antigen.

3. An isolated nucleic acid comprising a nucleotide sequence that encodes an alpha and a beta peptide of a T cell receptor, which binds a hepatitis D virus (HDV) antigen, wherein said HDV antigen is present on HDV or an HDV infected cell, and wherein T cells that express said T cell receptor inhibit HDV replication with or without killing the cell.

4. An isolated nucleic acid comprising a nucleotide sequence that encodes an alpha and a beta peptide of a T cell receptor, which binds a hepatitis D virus (HDV) antigen, wherein said HDV antigen is present on HDV or an HDV infected cell, and wherein T cells that express said T cell receptor inhibit HDV replication with or without killing the cell through the interaction with the HLA-A2 molecule loaded with a peptide from the HDV antigen.

5. The isolated nucleic acid of any one of alternatives 1-4, further comprising a nucleotide sequence encoding a cleavage sequence between said nucleotide sequence encoding the alpha and beta peptide.

6. The isolated nucleic acid of alternative 5, wherein said cleavage sequence is selected from the group consisting of porcine teschovirus-1 2A (P2A), foot-and-mouth disease virus (FMDV) 2A (F2A), equine rhinitis A virus (ERAV) 2A (E2A) and Thosea asigna virus 2A (T2A), wherein each cleavage sequence can be modified to include a GSG (glycine-serine-glycine) motif at the N-terminus.

7. A method of generating a nucleic acid encoding an alpha and/or beta chain of a cell receptor that is specific for a hepatitis D antigen (HDAg) comprising:
immunizing mice, such as HLA-A2 transgenic mice, with a nucleic acid comprising a nucleotide sequence encoding an HDAg;
isolating T cells from said immunized mice;
fusing said isolated cells with an immortalized cell, such as immortalized BW thymoma cells lacking a T cell receptor;
isolating a T cell hybridoma that is specific for or that binds to said HDAg;
isolating RNA, such as mRNA, encoding a T cell receptor alpha and/or beta chain from said T cell hybridoma; and
generating cDNA from said RNA, such as by reverse transcription.

8. The method of alternative 7, further comprising introducing said cDNA generated from said RNA into a plasmid, such as an expression plasmid.

9. The method of alternative 8, further comprising cloning said plasmid.

10. The method of anyone of alternatives 7-9 further comprising introducing a cleavage sequence between a cDNA encoding a T cell receptor alpha chain and a cDNA encoding a T cell receptor beta chain such that a fusion nucleic acid encoding a T cell receptor alpha chain, cleavage site and a T cell receptor beta chain is generated.

11. The method of alternative 10, wherein said cleavage sequence is selected from the group consisting of porcine teschovirus-1 2A (P2A), foot-and-mouth disease virus (FMDV) 2A (F2A), equine rhinitis A virus (ERAV) 2A (E2A) and Thosea asigna virus 2A (T2A), Wherein each cleavage sequence can be modified to include a GSG (glycine-serine-glycine) motif at the N-terminus.

12. The method of anyone of alternatives 7-11 further comprising:
isolating T cells from an individual, such as a patient infected with HDV or a patient that is at risk of becoming infected with HDV; and
introducing said cDNA or RNA encoding said alpha and/or beta chain of a T cell receptor that is specific for said HDAg into said isolated T cells so as to obtain modified cells.

13. The method of alternative 12, further comprising introducing said modified T cells into an individual, such as the individual from which said T cells were isolated.

14. The method of alternative 13, further comprising providing said individual an additional HBV and/or HDV medicament and/or therapy, such as a peptide or DNA-based HBV vaccine (e.g., a HBsAg-based HBV vaccine) and/or PEG-IFN.

15. Use of modified T cells obtained by the method of alternative 12 to prepare a medicament for the treatment, inhibition, and/or prevention of HDV infection or diseases associated therewith such as, chronic HBV-HDV co-infection, liver disease (e.g., HDV-mediated liver cirrhosis), and liver cancer (e.g., hepatocellular cancer (HCC)).

16. The use according to alternative 15, further comprising providing said individual an additional HBV and/or HDV medicament and/or therapy, such as a peptide or DNA-based HBV vaccine (e.g., a HBsAg-based HBV vaccine) and/or PEG-IFN.

17. Modified T cells obtained by the method of alternative 12 for use in treating, inhibiting, or preventing HDV infection of a disease associated therewith, such as chronic HBV-HDV co-infection, liver disease (e.g., HDV-mediated liver cirrhosis), and liver cancer (e.g., hepatocellular cancer (HCC)).

18. The modified T cells of alternative 17 for use in treating, inhibiting, or preventing HDV infection of a disease associated therewith, such as chronic HBV-HDV co-infection, liver disease (e.g., HDV-mediated liver cirrhosis), and liver cancer (e.g., hepatocellular cancer (HCC)) further comprising the provision of an additional HBV and/or HDV medicament and/or therapy, such as a peptide or DNA-based HBV vaccine (e.g., a HBsAg-based HBV vaccine) and/or PEG-IFN.

19. A method of inhibiting an HDV infection, HBV-HDV co-infection, HDV-mediated liver cirrhosis, or hepatocellular cancer (HCC) in a patient infected with HDV comprising:
immunizing HLA-A2 transgenic mice with a nucleic acid comprising a nucleotide sequence encoding an HDAg;
   isolating T cells from said immunized mice;
   fusing said isolated T cells with immortalized BW thymoma cells lacking a T cell receptor;
   isolating a T cell hybridoma that is specific for or that binds to said HDAg;
   isolating mRNA encoding a T cell receptor alpha and beta chains from said T cell hybridoma;
   generating cDNA from said mRNA by reverse transcription;
   introducing said cDNA generated from said mRNA into one or more plasmids;
   introducing a cleavage sequence between a cDNA encoding a T cell receptor alpha chain and a cDNA encoding a T cell receptor beta chain such that a fusion nucleic acid encoding a T cell receptor alpha chain, cleavage site and a T cell receptor beta chain is generated;
   isolating T cells from said patient;
   introducing said cDNA or RNA encoding said alpha and beta chain of a T cell receptor that is specific for said HDAg into said isolated T cells so as to obtain modified T cells; and
introducing said modified T cells into said patient.

20. The method of alternative 19, further comprising providing said patient an additional HBV and/or HDV medicament and/or therapy, such as a peptide or DNA-based HBV vaccine (e.g., a HBsAg-based HBV vaccine) and/or PEG-IFN.

21. The method of anyone of alternatives 19 or 20, wherein said cleavage sequence is selected from the group consisting of porcine teschovirus-1 2A (P2A), foot-and-mouth disease virus (FMDV) 2A (F2A), equine rhinitis A virus (ERAV) 2A (F2A) and Thosea asigna virus 2A (T2A), wherein each cleavage sequence can be modified to include a GSG (glycine-serine-glycine) motif at the N-terminus.

22. The isolated nucleic acids of any one of alternatives 1-6 for use in inhibiting, ameliorating, treating, or preventing an HDV infection, HBV-HDV co-infection, HDV-mediated liver cirrhosis, or hepatocellular cancer (HCC) in a patient infected with HDV.

23. The isolated nucleic acids of any one of alternatives 1-6 for use as a medicament.

24. Modified T cells comprising any one of the isolated nucleic acids of alternatives 1-6.

25. The modified T cells of alternative 24 for use as a medicament, such as for the treatment, inhibition, amelioration, or prevention of an HDV infection, HBV-HDV co-infection, HDV-mediated liver cirrhosis, or hepatocellular cancer (HCC).

26. The isolated nucleic acids or modified T cells of alternatives 23-25, further comprising providing an additional HBV and/or HDV medicament and/or therapy, such as a peptide or DNA-based HBV vaccine (e.g., a HBsAg-based HBV vaccine) and/or PEG-IFN.

### DETAILED DESCRIPTION OF THE INVENTION

Several approaches to treat, inhibit or prevent HDV infections by redirecting T cells to HDV and HDV infected cells are contemplated. In several alternatives, genes encoding a molecule that specifically binds to human leucocyte antigen (HLA)-A2 loaded with a HDV derived peptide are introduced into the T cells of individuals. The modified T cells obtained by this approach are then introduced into the patient (e.g., patients infected with HDV or patients that are at risk of becoming infected with HDV) and the modified T cells will then recognise and bind to the patient's cells that are infected by the HDV virus. Once the modified T cells bind to the patient's infected cells or HDV virus, the modified T cells will block HDV replication and/or kill the HDV infected cells thereby treating, inhibiting, or preventing the HDV infection in the individual or diseases associated therewith including, but not limited to, chronic HBV-HDV co-infection, liver disease (e.g., HDV-mediated liver cirrhosis), and/or liver cancer (e.g., hepatocellular cancer (HCC)).

### Generation Of Molecules That Recognize HDV In The Context Of An Hla Molecule, Introduction Into Patient's T Cells, And The Redirection Of The Modified T Cells In Patients To HDV And/Or HDV Infected Cells

Mice, for example HLA-2, or other human HLA, transgenic mice, are immunized with DNA plasmids encoding the hepatitis D antigen (HDAg). T cells specific for HDAg will then be isolated from the mice and these T cells will be fused with an immortalized cell line (e.g., a BW thymoma lacking its own T cell receptor). HDV-specific T cell hybridomas will then be identified and isolated. Thereafter, mRNA encoding the T cell receptor (TCR) alpha and beta chains will be isolated and reverse transcription will be used to generate cDNA. The cDNA will then be introduced into plasmids (e.g., expression plasmids) and cloned. In some alternatives, the expression plasmids comprising the alpha and beta chain pairs are introduced into T cells isolated from individuals (e.g., patients infected with HDV or patients that are at risk of becoming infected with HDV). These individuals may also be provided before, during, or after receiving the modified T cells a conventional HBV and/or HDV medicament and/or therapy including, but not limited to, a peptide or DNA-based HBV vaccine (e.g., a HBsAg-based HBV vaccine) and/or PEG-IFN.

In preferred alternatives, the nucleic acids encoding the alpha and beta chains are fused in cis and separated by a nucleic acid sequence encoding a cleavage site such that a fusion nucleic acid encoding an alpha chain, cleavage site, and beta chain is obtained. The fusion nucleic acid encoding the alpha chain, cleavage site, and beta chain will be introduced into a single expression vector and the expression vector comprising the nucleic acid encoding the alpha chain, cleavage site, and beta chain will be introduced into T cells isolated from individuals (e.g., patients infected with HDV or patients that are at risk of becoming infected with HDV). The modified T cells comprising the nucleic acid encoding the alpha chain, cleavage site, and beta chain will then be introduced into the individuals from which the T cells were obtained so as to treat, inhibit, and/or prevent HDV infection. Preferred nucleic acids encoding cleavage sites that can be used in these alternatives include, but are not limited to, self-cleavage domains, also referred to as “self-cleavage 2A peptide sequences” such as porcine teschovirus-1 2A (P2A), foot-and-mouth disease virus (FMDV) 2A (F2A), equine rhinitis A virus (ERAV) 2A (E2A) and Thosea asigna virus 2A (T2A), wherein each self-cleavage sequence can be modified to include a GSG (glycine-serine-glycine) motif at the N-terminus, which is contemplated to improve cleavage efficiency. The resulting fusion gene encoding the alpha chain, cleavage site, and beta chain will produce a functional T cell receptor that will recognize HLA-A2 molecules loaded with a HDV-derived peptide.

Although the invention has been described with reference to alternatives and examples, it should be understood that various modifications can be made without departing from the spirit of the invention.

1. An isolated nucleic acid comprising a nucleotide sequence that encodes an alpha and/or a beta peptide, which forms a part of or a complete T cell receptor, which binds a hepatitis D virus (HDV) antigen, wherein said HDV antigen is present on HDV or an HDV infected cell, and wherein T cells that express said T cell receptor inhibit HDV replication with or without killing the cell, wherein the isolated nucleic acid further comprises a nucleotide sequence that encodes a cleavage sequence between said nucleotide sequence encoding the alpha and beta peptide.

2-26. (canceled)

27. The isolated nucleic acid of claim 1, wherein said cleavage sequence is selected from the group consisting of porcine teschovirus-1 2A (P2A), foot-and-mouth disease virus (FMDV) 2A (F2A), equine rhinitis A virus (ERAV) 2A (E2A) and Thosea asigna virus 2A (T2A), wherein each cleavage sequence can be modified to include a GSG (glycine-serine-glycine) motif at the N-tenninus.

28. A method of generating a nucleic acid encoding an alpha and/or beta chain of a T cell receptor that is specific for a hepatitis D antigen (HDAg) comprising:
immunizing mice, such as HLA-A2 transgenic mice, with a nucleic acid comprising a nucleotide sequence encoding an HDAg;
isolating T cells from said immunized mice;
fusing said isolated T cells with an immortalized cell, such as immortalized BW thymoma cells lacking a T cell receptor;
isolating a T cell hybridoma that is specific for or that binds to said HDAg;
isolating RNA, such as mRNA, encoding a T cell receptor alpha and/or beta chain from said T cell hybridoma; and
generating cDNA from said RNA, such as by reverse transcription.

29. The method of claim 28, further comprising introducing said cDNA generated from said RNA into a plasmid, such as an expression plasmid.

30. The method of claim 30, further comprising cloning said plasmid.

31. The method of claim 28, further comprising introducing a cleavage sequence between a cDNA encoding a T cell receptor alpha chain and a cDNA encoding a T cell receptor beta chain such that a fusion nucleic acid encoding a T cell receptor alpha chain, cleavage site and a T cell receptor beta chain is generated.

32. The method of claim 29, wherein said cleavage sequence is selected from the group consisting of porcine teschovirus-1 2A (P2A), foot-and-mouth disease virus (FMDV) 2A (F2A), equine rhinitis A virus (ERAV) 2A (E2A) and Thosea asigna virus 2A (T2A), wherein each cleavage sequence can be modified to include a GSG (glycine-serine-glycine) motif at the N-terminus.

33. The method of claim 28 further comprising:
isolating T cells from an individual, such as a patient infected with HDV or a patient that is at risk of becoming infected with HDV; and
introducing said cDNA or RNA encoding said alpha and/or beta chain of a T cell receptor that is specific for said HDAg into said isolated T cells so as to obtain modified T cells.

34. The method of claim 33, further comprising introducing said modified T cells into an individual, such as the individual from which said T cells were isolated.

35. The method of claim 34, further comprising providing said individual an additional HBV and/or HDV medicament and/or therapy, such as a peptide or DNA-based HBV vaccine (e.g., a HBsAg-based HBV vaccine) and/or PEG-IFN.

36. A method of inhibiting an HDV infection, HBV-HDV co-infection, HDV-mediated mediated liver cirrhosis, or hepatocellular cancer (HCC) in a patient infected with HDV comprising:
immunizing HLA-A2 transgenic mice with a nucleic acid comprising a nucleotide sequence encoding an HDAg;
isolating T cells from said immunized mice;
fusing said isolated T cells with immortalized BW thymoma cells lacking a T cell receptor;
isolating a T cell hybridoma that is specific for or that binds to said HDAg;
isolating mRNA encoding a T cell receptor alpha and beta chains from said T cell hybridoma;
generating cDNA from said mRNA by reverse transcription;
introducing said cDNA generated from said mRNA into one or more plasmids;
introducing a cleavage sequence between a cDNA encoding a T cell receptor alpha chain and a cDNA encoding a T cell receptor beta chain such that a fusion nucleic acid encoding a T cell receptor alpha chain, cleavage site and a T cell receptor beta chain is generated;
isolating T cells from said patient;
introducing said cDNA or RNA encoding said alpha and beta chain of a T cell receptor that is specific for said HDAg into said isolated T cells so as to obtain modified T cells; and
introducing said modified T cells into said patient.

37. The method of claim 36, further comprising providing said patient an additional HBV and/or HDV medicament and/or therapy, such as a peptide or DNA-based HBV vaccine (e.g., a HBsAg-based HBV vaccine) and/or PEG-IFN.

38. The method of claim 36, wherein said cleavage sequence is selected from the group consisting of porcine teschovirus-1 2A (P2A), foot-and-mouth disease virus (FMDV) 2A (F2A), equine rhinitis A virus (ERAV) 2A (E2A) and Thosea asigna virus 2A (T2A), wherein each cleavage sequence can be modified to include a GSG (glycine-serine-glycine) motif at the N-terminus.
